(19) United States
(12) Patent Application Publication
(10) Pub. No.: US 2024/0123434 A1
(43) Pub. Date: Apr. 18, 2024

(54) MULTIFUNCTIONAL CATALYST, METHOD FOR PRODUCING THE SAME, AND METHOD FOR USING THE SAME

(71) Applicant: NAN YA PLASTICS CORPORATION, TAIPEI (TW)

(72) Inventors: TE-CHAO LIAO, TAIPEI (TW); WEI-SHENG CHENG, TAIPEI (TW); YU-LIN LI, Taoyuan City (TW)

(21) Appl. No.: 18/079,077

(22) Filed: Dec. 12, 2022

(30) Foreign Application Priority Data
Sep. 27, 2022 (TW) 111136451

(51) Int. Cl. B01J 31/02; B01J 21/02; B01J 23/78; B01J 31/28; B01J 35/02; B01J 37/02; B01J 37/08; B01J 37/16; C07C 67/00; C08J 11/24

(52) U.S. Cl. CPC B01J 31/0284; B01J 21/02; B01J 23/78; B01J 31/0298; B01J 31/28; B01J 35/026; B01J 37/0219; B01J 37/0221; B01J 37/08; B01J 37/16; C07C 67/00; C08J 11/24; B01J 2231/005; B01J 2531/002; C08J 2367/02

(57) ABSTRACT

A multifunctional catalyst, a method for producing the same, and a method for using the same are provided. The multifunctional catalyst is applicable for recycling a polyester fabric. The multifunctional catalyst includes a carrier, and a first functional ionic liquid and a second functional ionic liquid that are grafted on the carrier. The carrier is an inorganic composite powder material, and is composed of following chemical components: C: Na—Ni/Al₂O₃. In a process of recycling the polyester fabric, the multifunctional catalyst simultaneously decolorizes and depolymerizes the polyester fabric. The first functional ionic liquid is used to decolorize the polyester fabric, and the second functional ionic liquid is used to depolymerize the polyester fabric.

### CROSS-REFERENCE TO RELATED PATENT APPLICATION

This application claims the benefit of priority to Taiwan Patent Application No. 111136451, filed on Sep. 27, 2022. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a catalyst, and more particularly to a multifunctional catalyst capable of recycling a polyester fabric, a method for producing the same, and a method for using the same.

### BACKGROUND OF THE DISCLOSURE

PET closed-loop chemical recycling technology has undergone rapid development in recent years. In the chemical recycling technology, solvent-assisted chemical degradation, biotechnology-assisted chemical degradation, and microwave-assisted chemical degradation have all reached the trial production stage of the development stages. The chemical recycling technology can be used to process different types of waste polyester materials, such as waste polyester bottle flakes or waste polyester textile fabrics.

For recycling of the waste polyester textile fabrics, depolymerization and decolorization of the waste polyester textile fabrics are separately conducted in different operation processes in most of the existing chemical recycling technologies. Furthermore, a catalyst is difficult to be recycled, such that a high cost is incurred in the recycling operation.

### SUMMARY OF THE DISCLOSURE

In response to the above-referenced technical inadequacies, the present disclosure provides an environmentally friendly multifunctional catalyst for recycling a polyester fabric, a method for producing the same, and a method for using the same.

In one aspect, the present disclosure provides a multifunctional catalyst that is applicable for recycling a polyester fabric. The multifunctional catalyst includes a carrier, a first functional ionic liquid, and a second functional ionic liquid. The carrier is an inorganic composite powder material composed of the following chemical components: C: Na—Ni/Al₂O₃. The first functional ionic liquid is grafted on the carrier. The second functional ionic liquid is grafted on the carrier. In a process of recycling the polyester fabric, the multifunctional catalyst can simultaneously decolorize and depolymerize the polyester fabric, the first functional ionic liquid is used to decolorize the polyester fabric and the second functional ionic liquid is used to depolymerize the polyester fabric.

In certain embodiments, the inorganic composite powder material is composed of a sodium-nickel-to-alumina composite and a carbon-absorbing material, the inorganic composite powder material absorbs carbon by using a nickel atom end of the inorganic composite powder material, and the inorganic composite powder material is configured to decolorize the polyester fabric by using the carbon absorbed at the nickel atom end.

In certain embodiments, the first functional ionic liquid is an imidazole ionic liquid, and is at least one material selected from a group consisting of [C4mim][PF6], [C6mim][PF6], [C6mim][BF4], and [C8mim][PF6].

In certain embodiments, the second functional ionic liquid is a salt composed of a cation and an anion, the cation is at least one material selected from a group consisting of imidazolium cation, pyridium cation, quaternary phosphonium cation, and quaternary ammonium cation; and the anion is at least one material selected from a group consisting of CI⁻, Br⁻, I⁻, AlCl₄⁻, AlBr₄⁻, AlI₄⁻, CF₃COO⁻, CH₃COO⁻, CF₃SO₃⁻, SCN⁻, (CF₃SO₂)₂N⁻, (CF₃SO₂⁻)₃C⁻, and C₆H₄(OH)(COO⁻).

In certain embodiments, the imidazolium cation has a chemical structure as shown by the following formula (1):

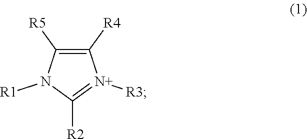

the pyridium cation has a chemical structure as shown by the following formula (2):

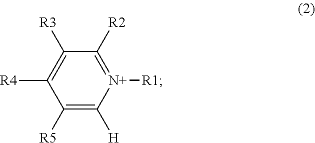

the quaternary phosphonium cation has a chemical structure as shown by the following formula (3):

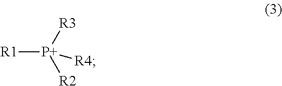

and

the quaternary ammonium cation has a chemical structure as shown by the following formula (4):

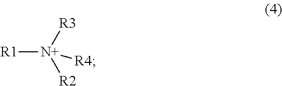

in which R1, R2, R3, R4, and R5 are each an alkane group, a halogenated hydrocarbyl group, a hydroxyl group, an aromatic group, or a heterocyclic hydrocarbyl group that are independent from each other.

In certain embodiments, based on a total weight of the inorganic composite powder material being 100 wt %, the content of the carbon component ranges from 10 wt % to 15 wt %.

In certain embodiments, an ionic liquid grafting ratio of the first functional ionic liquid to the second functional ionic liquid being grafted on the carrier ranges from 5% to 40%.

In certain embodiments, an ionic liquid grafting ratio of the first functional ionic liquid to the second functional ionic liquid being grafted on the carrier ranges from 5% to 25%.

In certain embodiments, a weight of the first functional ionic liquid capable of decolorization is between 2 times and 10 times a weight of the second functional ionic liquid capable of depolymerization.

In another aspect, the present disclosure provides a method for producing a multifunctional catalyst. The method includes: providing a first inorganic composite powder material that is composed of the following chemical components: C: Na—Ni/Al₂O₃; implementing a reduction operation, including: using a fixed bed reactor to introduce carbon dioxide gas into the first inorganic composite powder material under a reaction condition, so as to reduce the carbon dioxide gas into a carbon component that is absorbed at a nickel atom end of the first inorganic composite powder material; implementing a sintering operation, including: under a sintering condition, performing lattice rearrangement for the first inorganic composite powder material absorbed with the carbon component at the nickel atom end, so as to obtain a second inorganic composite powder material that is composed of the following chemical components: C: Na—Ni/Al₂O₃; and implementing a grafting operation, including: respectively reacting a first functional ionic liquid and a second functional ionic liquid with a siloxane coupling agent, and having the first functional ionic liquid and the second functional ionic liquid to be grafted on the second inorganic composite powder material through the siloxane coupling agent, so as to obtain the multifunctional catalyst.

In certain embodiments, the first functional ionic liquid is an imidazole ionic liquid, and is at least one material selected from a group consisting of [C4mim][PF6], [C6mim][PF6], [C6mim][BF4], and [C8mim][PF6].

In certain embodiments, the second functional ionic liquid is a salt composed of a cation and an anion, the cation is at least one material selected from a group consisting of imidazolium cation, pyridium cation, quaternary phosphonium cation, and quaternary ammonium cation; and the anion is at least one material selected from a group consisting of CI⁻, Br⁻, I⁻, AlCl₄⁻, AlBr₄⁻, AlI₄⁻, CF₃COO⁻, CH₃COO⁻, CF₃SO₃⁻, SCN⁻, (CF₃SO₂)₂N⁻, (CF₃SO₂⁻)₃C⁻, and C₆H₄(OH)(COO⁻).

In yet another aspect, the present disclosure provides a method for using a multifunctional catalyst. The method includes: providing a polyester fabric that is a dyed polyester fabric; providing a multifunctional catalyst that includes a carrier, and a first functional ionic liquid and a second functional ionic liquid that are grafted on the carrier, where the carrier is an inorganic composite powder material that is composed of the following chemical components: C: Na—Ni/Al₂O₃; mixing the polyester fabric, the multifunctional catalyst, and a chemical depolymerization solution so that the first functional ionic liquid decolorizes the polyester fabric and the second functional ionic liquid depolymerizes the polyester fabric, so as to obtaining a depolymerized product after decolorization and depolymerization, where the depolymerized product contains bis(2-hydroxyethyl) terephthalate (BHET); and separating the multifunctional catalyst from the BHET.

Therefore, in the multifunctional catalyst, the method for producing the same and the method for using the same provided by the present disclosure, by the special selection of materials for the carrier and by the first functional ionic liquid and the second functional ionic liquid being grafted on the carrier, the multifunctional catalyst has the functions of both depolymerization and decolorization, and can be easily recycled. Through the special material design for the multifunctional catalyst provided by the present disclosure, the polyester fabric can be decolorized and depolymerized in a single operation process, such that the issue of an excessively high recycling cost incurred in the existing chemical recycling technology can be addressed.

Further, the inorganic composite powder material can also decolorize a dye in the polyester fabric by using the carbon absorbed at its nickel atom end, thereby improving the decolorization efficiency of the multifunctional catalyst.

In addition, because the inorganic composite powder material is composed of a sodium-nickel-to-alumina composite, the multifunctional catalyst may be recycled by using a magnetic substance (for example, a magnet), or may be recycled by filtering.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic diagram of a chemical structure of a multifunctional catalyst in one embodiment of the present disclosure;

FIG. 2 is a flowchart of a method for producing a multifunctional catalyst in one embodiment of the present disclosure; and

FIG. 3 is a flowchart of a method for using a multifunctional catalyst in one embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

### Multifunctional Catalyst

One embodiment of the present disclosure provides a multifunctional catalyst that is applicable for recycling a polyester fabric. In a process of recycling the polyester fabric, the multifunctional catalyst can decolorize and depolymerize the polyester fabric in the same operation process, and the multifunctional catalyst can be easily recycled, thereby reducing the recycling cost. The polyester particularly refers to polyethylene terephthalate (PET).

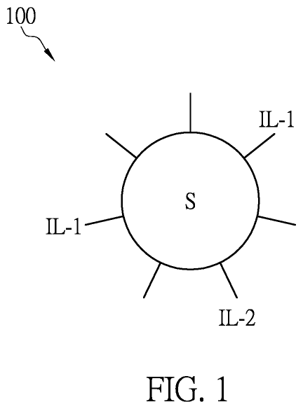

As shown in FIG. 1, a multifunctional catalyst 100 includes: a carrier S, and a first functional ionic liquid IL-1 and a second functional ionic liquid IL-2 that are grafted on the carrier S.

The carrier S is an inorganic composite powder material that is composed of the following chemical components: C: Na—Ni/Al₂O₃. In other words, the inorganic composite powder material is composed of a sodium-nickel-to-alumina composite (Na—Ni/Al₂O₃) and a carbon-absorbing material, in which the inorganic composite powder material absorbs carbon (C:) with its nickel atom end.

The first functional ionic liquid IL-1 and the second functional ionic liquid IL-2 are grafted on the carrier S through chemical covalent bonding. The first functional ionic liquid IL-1 is used to decolorize the polyester fabric in the process of recycling the polyester fabric, and the second functional ionic liquid IL-2 is used to depolymerize the polyester fabric in the process of recycling the polyester fabric. That is to say, the first functional ionic liquid IL-1 and the second functional ionic liquid IL-2 can decolorize and depolymerize the polyester fabric in the same operation process.

It should be noted that, the inorganic composite powder material can also decolorize a dye in the polyester fabric by using the carbon absorbed at its nickel atom end, thereby improving the decolorization efficiency of the multifunctional catalyst.

In addition, because the inorganic composite powder material is composed of a sodium-nickel-to-alumina composite (Na—Ni/Al₂O₃), the multifunctional catalyst 100 may be recycled by using a magnetic substance (e.g., a magnet) or may also be recycled by filtering.

Furthermore, the first functional ionic liquid IL-1 capable of decolorization may be, for example, an imidazole ionic liquid.

In the embodiments of the present disclosure, the first functional ionic liquid is at least one material selected from a group consisting of [C4mim][PF6], [C6mim][PF6], [C6mim][BF4], and [C8mim][PF6]. The first functional ionic liquid can be used to eliminate anionic dyes, such as methyl orange, Eosin yellowish, and orange G solution; and the first functional ionic liquid has good decolorization efficiency for these anionic dyes in an aqueous environment with a suitable pH value. Furthermore, [PF6] or [BF4] in the foregoing ionic liquid has the ability of eliminating the dyes.

Moreover, the second functional ionic liquid IL-2 capable of depolymerization may be a salt composed of, for example, a cation and an anion. The cation of the second functional ionic liquid IL-2 is at least one material selected from a group consisting of imidazolium cation, pyridium cation, quaternary phosphonium cation, and quaternary ammonium cation; and the cation is capable of depolymerizing the polyester.

Table 1 shows chemical structures of cations of the second functional ionic liquid, in which the imidazolium cation has a chemical structure shown by formula (1), the pyridium cation has a chemical structure shown by formula (2), the quaternary phosphonium cation has a chemical structure shown by formula (3), and the quaternary ammonium cation has a chemical structure shown by formula (4).

TABLE 1

| Cation type | Chemical structure |
|---|---|
| Imidazolium cation | 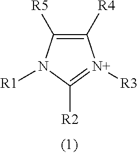 |
| Pyridium cation | 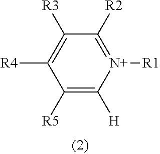 |
| Quaternary phosphonium cation | 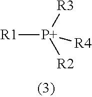 |
| Quaternary ammonium cation | 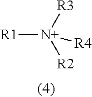 |

The substituents, R1, R2, R3, R4, and R5, in the foregoing chemical structures are each an alkane group, a halogenated hydrocarbyl group, a hydroxyl group, an aromatic group, or a heterocyclic hydrocarbyl group that are independent from each other (i.e., the substituents may be the same or different), in which the number of carbons in organic substituent groups of aliphatic chains of the substituents R1, R2, R3, R4, and R5 ranges from 1 to 14.

The anion of the second functional ionic liquid IL-2 is at least one material selected from a group consisting of CI⁻, Br⁻, I⁻, AlCl₄⁻, AlBr₄⁻, AlI₄⁻, CF₃COO⁻, CH₃COO⁻, CF₃SO₃⁻, SCN⁻, (CF₃SO₂)₂N⁻, (CF₃SO₂⁻)₃C⁻, and C₆H₄(OH)(COO⁻).

In certain embodiments of the present disclosure, based on a total weight of the inorganic composite powder material (C: Na—Ni/Al₂O₃) being 100 wt %, a content of the carbon component is from 10 wt % to 15 wt % of the total weight. Therefore, the carbon absorbed at the nickel atom end can enable the inorganic composite powder material to have a decolorization effect.

If the content of the carbon component is too high, the carbon component cannot be continuously absorbed on the nickel atom end due to having a saturated concentration. Conversely, if the content of the carbon component is too low, the carbon cannot provide a sufficient decolorization effect due to having a low concentration.

In certain embodiments of the present disclosure, the weight of the carrier S (C: Na—Ni/Al₂O₃) of the multifunctional catalyst 100 is defined as a first weight. A weight sum of the first functional ionic liquid IL-1 and the second functional ionic liquid IL-2 of the multifunctional catalyst 100 is defined as a second weight. Furthermore, a weight ratio of the first weight to the second weight ranges from 1:0.05 to 1:0.40, and preferably from 1:0.05 to 1:0.25. That is to say, the weight sum of the first functional ionic liquid IL-1 and the second functional ionic liquid IL-2 is between 5% and 40% (preferably between 5% and 25%) of the weight of the carrier S. In other words, an ionic liquid grafting ratio of the first functional ionic liquid IL-1 to the second functional ionic liquid IL-2 being grafted on the carrier S ranges from 5% to 40% (preferably ranges from 5% to 25%).

In certain embodiments of the present disclosure, a weight ratio between the first functional ionic liquid IL-1 capable of decolorization to the second functional ionic liquid IL-2 capable of depolymerization ranges from 2:1 to 10:1, and is preferably 4:1. That is to say, the weight of the first functional ionic liquid IL-1 capable of decolorization is between 2 times and 10 times (preferably 4 times) the weight of the second functional ionic liquid IL-2 capable of depolymerization, but the present disclosure is not limited thereto.

Accordingly, by the special selection of materials for the carrier S and by the first functional ionic liquid IL-1 and the second functional ionic liquid IL-2 grafted on the carrier S, the multifunctional catalyst 100 in an embodiment of the present disclosure can have the functions of both depolymerization and decolorization, and can be easily recycled. By the particular material design for the multifunctional catalyst 100 provided in the embodiment of the present disclosure, the polyester fabric can be decolorized and depolymerized in a single operation process, such that the issue of an excessively high recycling cost incurred in the existing chemical recycling technology can be addressed.

### Method for Producing the Multifunctional Catalyst

The above description is related to the material characteristics of the multifunctional catalyst in the embodiments of the present disclosure. In the following description, a method for producing the multifunctional catalyst will be illustrated according to one embodiment of the present disclosure.

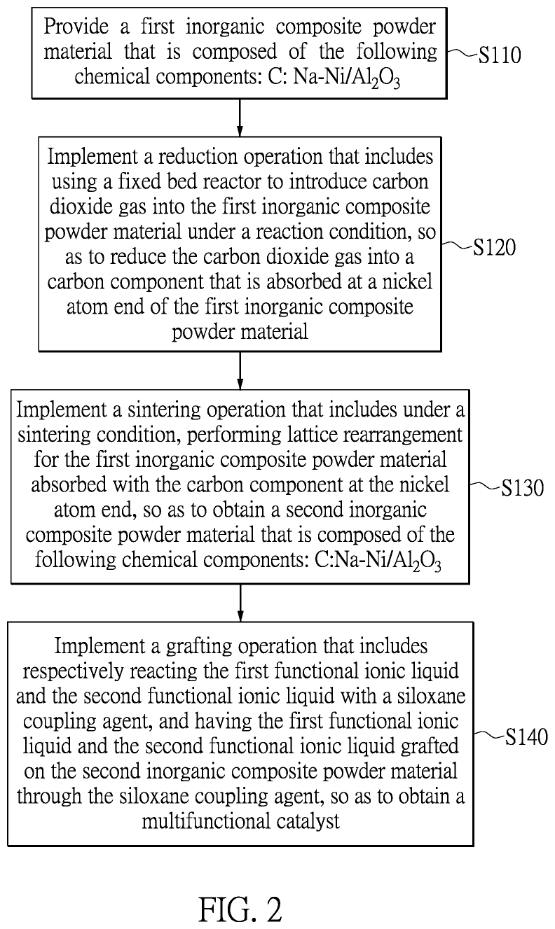

As shown in FIG. 2, the manufacturing method of the multifunctional catalyst includes steps S110, S120, S130, and S140. It should be noted that, a sequence and a practical operation of the steps described in this embodiment can be adjusted as required, and the present disclosure is not limited to the description in this embodiment.

Step S110 includes: providing a first inorganic composite powder material that is composed of the following chemical components: Na—Ni/Al₂O₃.

Step S120 includes: implementing a reduction operation. The reduction operation includes: using a fixed bed reactor to introduce carbon dioxide gas into the first inorganic composite powder material under reaction conditions of a reaction temperature and a reaction time, so as to reduce the carbon dioxide gas into a carbon component that is absorbed at a nickel atom end of the first inorganic composite powder material (Na—Ni/Al₂O₃), in which the reaction temperature ranges from 400° C. to 800° C. (preferably from 400° C. to 600° C.) and the reaction time ranges from 2 hours to 72 hours (preferably from 12 hours to 36 hours).

Step S130 includes: implementing a sintering operation. The sintering operation includes: under sintering conditions of a sintering temperature and a sintering time, performing lattice rearrangement for the first inorganic composite powder material absorbed with the carbon component at the nickel atom end, so as to obtain a second inorganic composite powder material that is composed of the following chemical components: C: Na—Ni/Al₂O₃. The sintering temperature ranges from 500° C. to 800° C. (preferably from 500° C. to 700° C.), and the sintering time ranges from 1 hour to 2 hours (preferably from 12 hours to 48 hours).

Step S140 includes: implementing a grafting operation. The grafting operation includes: respectively reacting the first functional ionic liquid and the second functional ionic liquid with a siloxane coupling agent, and having the first functional ionic liquid and the second functional ionic liquid grafted on the second inorganic composite powder material through the siloxane coupling agent, so as to obtain the multifunctional catalyst.

### Method for Using the Multifunctional Catalyst

The above description is related to the method for producing the multifunctional catalyst in the embodiments of the present disclosure. In the following description, a method for using the multifunctional catalyst will be illustrated according to one embodiment of the present disclosure.

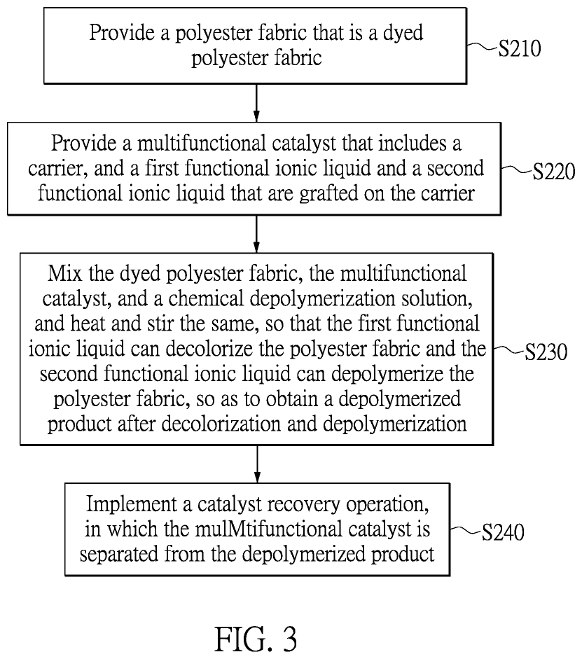

As shown in FIG. 3, the method for using the multifunctional catalyst includes steps S210, S220, S230, and S240. It should be noted that, a sequence and a practical operation of the steps described in this embodiment can be adjusted as required, and the present disclosure is not limited to the description in this embodiment.

Step S210 includes: providing a polyester fabric that is a dyed polyester fabric.

Step S220 includes: providing a multifunctional catalyst, in which the multifunctional catalyst includes a carrier and a first functional ionic liquid and a second functional ionic liquid that are grafted on the carrier.

The carrier is an inorganic composite powder material that is composed of the following chemical components: C: Na—Ni/Al₂O₃. From another perspective, the inorganic composite powder material is composed of a sodium-nickel-to-alumina composite (Na—Ni/Al₂O₃) and a carbon-absorbing material, in which the inorganic composite powder material absorbs carbon (C:) with its nickel atom end.

The first functional ionic liquid may be, for example, an imidazole ionic liquid. The first functional ionic liquid is at least one material selected from a group consisting of [C4mim][PF6], [C6mim][PF6], [C6mim][BF4], and [C8mim] [PF6].

The cation of the second functional ionic liquid is at least one material selected from a group consisting of imidazolium cation, pyridium cation, quaternary phosphonium cation, and quaternary ammonium cation. The anion of the second functional ionic liquid is at least one material selected from a group consisting of CI⁻, Br⁻, I⁻, AlCl₄⁻, AlBr₄⁻, AlI₄⁻, CF₃COO⁻, CH₃COO⁻, CF₃SO₃⁻, SCN⁻, (CF₃SO₂)₂N⁻, (CF₃SO₂⁻)₃C⁻, and C₆H₄(OH)(COO⁻).

Step S230 includes: mixing the dyed polyester fabric, the multifunctional catalyst, and a chemical depolymerization solution according to a predetermined weight ratio, and heating and stirring the mixture, so that the first functional ionic liquid can decolorize the polyester fabric and the second functional ionic liquid can depolymerize the polyester fabric, so as to obtain a depolymerized product after decolorization and depolymerization. That is to say, the first functional ionic liquid and the second functional ionic liquid can decolorize and depolymerize the polyester fabric in the same operation process.

The dyed polyester fabric, the multifunctional catalyst, and the chemical depolymerization solution are mixed according to a pre-determined weight ratio of 10 to 30:0.05 to 1:60 to 90, but the present disclosure is not limited thereto.

The depolymerized product contains bis(2-hydroxyethyl) terephthalate (BHET), the multifunctional catalyst, and the chemical depolymerization solution, and the BHET is formed via degradation of the polyester fabric. Moreover, the chemical depolymerization solution may be ethylene glycol (EG), but the present disclosure is not limited thereto.

It should be noted that, the inorganic composite powder material can also decolorize a dye in the polyester fabric by using the carbon absorbed at its nickel atom end, thereby improving the decolorization efficiency of the multifunctional catalyst.

Step S240 includes: implementing a catalyst recycling operation, in which the multifunctional catalyst is separated from the BHET and the chemical depolymerization solution. The catalyst recycling operation may be implemented by, for example, magnetically attracting the multifunctional catalyst with a magnetic substance (e.g., a magnet), but the present disclosure is not limited thereto. The catalyst recycling operation may recover the multifunctional catalyst through filtering or centrifugation.

According to the foregoing configuration, the depolymerized product has a degradation efficiency (an efficiency of the polyester fabric being degraded into the BHET) of not less than 90% by a gel permeation chromatography (GPC) test; and the BHET has a value L of not less than 90, a value a ranging from -1 to 1, and a value b ranging from −5 to 5.

### Experimental Data and Test Results

Hereinafter, the content of the present disclosure is described in detail with reference to Examples 1 to 4. However, the following examples are provided only to aid in the understanding of the present disclosure, and the scope of the present disclosure is not limited to these examples.

Preparation steps of Example 1 include: a light-colored dyed polyester fabric being provided and placed in a chemical depolymerization solution (e.g., ethylene glycol); a multifunctional catalyst being placed in the chemical depolymerization solution to decolorize and depolymerize the light-colored dyed polyester fabric, in which the multifunctional catalyst includes a carrier and a first functional ionic liquid and a second functional ionic liquid that are grafted on the carrier.

The carrier is composed of C: Na—Ni/Al₂O₃. In Example 1, the material of the first functional ionic liquid is [C4mim][PF6], and in the second functional ionic liquid, the cation is imidazolium cation and the anion is CI⁻. An ionic liquid grafting ratio of the first functional ionic liquid to the second functional ionic liquid being grafted on the carrier is 20.1% (part by weight). In addition, a weight ratio between the first functional ionic liquid (for decolorization) and the second functional ionic liquid (for depolymerization) is 4:1. An addition weight of the multifunctional catalyst is 2% of the weight of the polyester fabric. A reaction time for depolymerization and decolorization is 2 hours. It should be noted that, for the first functional ionic liquid and the second functional ionic liquid, only one material combination is exemplified in Examples 1 to 4, but the present disclosure is not limited thereto. It can be understood by persons having ordinary skills in the art that, although other listed material types of the first functional ionic liquid and the second functional ionic liquid that have similar properties are not tested in the specification, similar technical effects can be deduced from the above-mentioned examples. That is, the first ionic liquid is capable of decolorization and the second ionic liquid is capable of depolymerization.

After testing, a depolymerization efficiency of Example 1 is 93%, that is, 93% of the polyester fabric is degraded into the BHET, which indicates that the preparation conditions in Example 1 provide a good depolymerization efficiency. With respect to the decolorization effect, the BHET in Example 1 has a value L of 80, a value a of 0.16, and a value b of 4.46, which indicates that the preparation conditions in Example 1 can enable the BHET to have a good hue.

The abovementioned depolymerization efficiency is obtained by GPC analysis and test. The depolymerization efficiency of the polyester is calculated via a calculation of: (initial mass of polyester−mass of unpolymerized polyester)/initial mass of polyester*100%. A Lab color space is a color-component space, and the Lab value is tested by using a spectrometer.

The preparation conditions of Example 2 are substantially identical to those of Example 1, and the difference lies in that an ionic liquid grafting ratio in Example 2 is 14.2%. In Example 2, a depolymerization efficiency is 92%, and the BHET has a value L of 83, a value a of −0.65, and a value b of 3.9. The preparation conditions of Example 3 are substantially identical to those of Example 1, and the difference lies in that an ionic liquid grafting ratio in Example 3 is 9.0%. In Example 3, a depolymerization efficiency is 91%, and the BHET has a value L of 85, a value a of −0.73, and a value b of 3.1. The preparation conditions of Example 4 are substantially identical to those of Example 1, and the difference lies in that an ionic liquid grafting ratio in Example 4 is 5.0%. In Example 4, a depolymerization efficiency is 91%, and the BHET has a value L of 86, a value a of 0.07, and a value b of 1.8. In general, as shown in Table 2, the preparation conditions in Examples 1 to 4 all have a good depolymerization efficiency (greater than 90%), and all have a good hue (L>80, a =−1 to 1, b=−5 to 5).

TABLE 2

| | | | Depolymerization | | | | | |
|---|---|---|---|---|---|---|---|---|
| | | Multifunctional | Catalyst | | | | | |
| Example | | catalyst | addition | | Depolymerization | Decolorization | | |
| No./preparation | | Ionic liquid | amount | Time | efficiency | effect (hue) | | |
| conditions | | grafting ratio (%) | (%) | (hours) | % | L | a | b |
| 1 | Light-colored | 20.1 | 2 | 2 | 93 | 80 | 0.16 | 4.46 |
| 2 | polyester | 14.2 | 2 | 2 | 92 | 83 | −0.65 | 3.9 |
| 3 | fabric | 9.0 | 2 | 2 | 91 | 85 | −0.73 | 3.1 |
| 4 | | 5 | 2 | 2 | 91 | 86 | 0.07 | 1.8 |

Then, as shown in Table 3, the multifunctional catalyst with the ionic liquid grafting ratio of 14.2% (in Example 2) is subjected to a catalyst depolymerization-recycing cycle test for 20 times, in which all the reaction times for depolymerization and decolorization are 2 hours. Under the foregoing conditions, the catalyst recycled each time can provide a depolymerization efficiency of not less than 90%, and the decolorization effects of the BHET can all meet the following standards: L>80, a=−1 to 1, and b=−5 to 5.

TABLE 3

| Catalyst | | | | | | |
|---|---|---|---|---|---|---|
| addition | Number | Depolymer- | Depolymer- | Decolorization | | |
| amount | of | ization | ization | effect (hue) | | |
| % | reuses | time (hours) | efficiency % | L | a | b |
| 2 | 2 | 2 | 91 | 83 | −0.19 | 3.63 |
| 2 | 5 | 2 | 92 | 83 | −0.28 | 3.89 |
| 2 | 20 | 2 | 91 | 83 | −0.60 | 4.03 |

### Beneficial Effects of the Embodiments

Therefore, in the multifunctional catalyst, the method for producing the same and the method for using the same provided by the present disclosure, by the special selection of materials for the carrier and by the first functional ionic liquid and the second functional ionic liquid being grafted on the carrier, the multifunctional catalyst has the functions of both depolymerization and decolorization, and can be easily recycled. Through the special material design for the multifunctional catalyst provided by the present disclosure, the polyester fabric can be decolorized and depolymerized in a single operation process, such that the issue of an excessively high recycling cost incurred in the existing chemical recycling technology can be addressed.

Further, the inorganic composite powder material can also decolorize a dye in the polyester fabric by using the carbon absorbed at its nickel atom end, thereby improving the decolorization efficiency of the multifunctional catalyst.

In addition, because the inorganic composite powder material is composed of a sodium-nickel-to-alumina composite, the multifunctional catalyst may be recycled by using of a magnetic substance (for example, a magnet) or may be recycled by filtering.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A multifunctional catalyst, applicable for recycling a polyester fabric, wherein the multifunctional catalyst comprises:
a carrier, wherein the carrier is an inorganic composite powder material that is composed of the following chemical components: C: Na—Ni/Al₂O₃;
a first functional ionic liquid grafted on the carrier; and
a second functional ionic liquid grafted on the carrier;
wherein in a process of recycling the polyester fabric, the multifunctional catalyst is able to simultaneously decolorize and depolymerize the polyester fabric, the first functional ionic liquid is used to decolorize the polyester fabric, and the second functional ionic liquid is used to depolymerize the polyester fabric.

2. The multifunctional catalyst according to claim 1, wherein the inorganic composite powder material is composed of a sodium-nickel-to-alumina composite and a carbon-absorbing material, the inorganic composite powder material absorbs carbon by using a nickel atom end of the inorganic composite powder material, and the inorganic composite powder material is configured to decolorize the polyester fabric by using the carbon absorbed at the nickel atom end.

3. The multifunctional catalyst according to claim 1, wherein the first functional ionic liquid is an imidazole ionic liquid, and is at least one material selected from a group consisting of [C4mim][PF6], [C6mim][PF6], [C6mim][BF4], and [C8mim][PF6].

4. The multifunctional catalyst according to claim 3, wherein the second functional ionic liquid is a salt composed of a cation and an anion, the cation is at least one material selected from a group consisting of imidazolium cation, pyridium cation, quaternary phosphonium cation, and quaternary ammonium cation, and the anion is at least one material selected from a group consisting of CI⁻, Br⁻, I⁻, AlCl₄⁻, AlBr₄⁻, AlI₄⁻, CF₃COO⁻, CH₃COO⁻, CF₃SO₃⁻, SCN⁻, (CF₃SO₂)₂N⁻, (CF₃SO₂⁻)₃C⁻, and C₆H₄(OH)(COO⁻).

5. The multifunctional catalyst according to claim 4, wherein the imidazolium cation has a chemical structure as shown by the following formula (1): 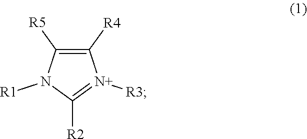
the pyridium cation has a chemical structure as shown by the following formula (2): 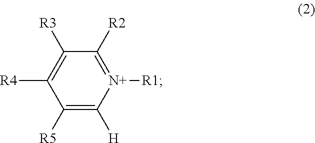
the quaternary phosphonium cation has a chemical structure as shown by the following formula (3): 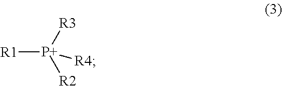
and
the quaternary ammonium cation has a chemical structure as shown by the following formula (4): 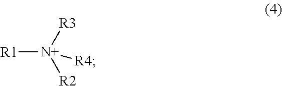
wherein R1, R2, R3, R4, and R5 are each an alkane group, a halogenated hydrocarbyl group, a hydroxyl group, an aromatic group, or a heterocyclic hydrocarbyl group that are independent from each other.

6. The multifunctional catalyst according to claim 2, wherein based on a total weight of the inorganic composite powder material being 100 wt %, the content of the carbon component ranges from 10 wt % to 15 wt %.

7. The multifunctional catalyst according to claim 1, wherein an ionic liquid grafting ratio of the first functional ionic liquid to the second functional ionic liquid being grafted on the carrier ranges from 5% to 40%.

8. The multifunctional catalyst according to claim 7, wherein the ionic liquid grafting ratio of the first functional ionic liquid to the second functional ionic liquid being grafted on the carrier ranges from 5% to 25%.

9. The multifunctional catalyst according to claim 7, wherein a weight of the first functional ionic liquid capable of decolorization is between 2 times and 10 times a weight of the second functional ionic liquid capable of depolymerization.

10. A method for producing a multifunctional catalyst, comprising:
providing a first inorganic composite powder material that is composed of the following chemical components: Na—Ni/Al₂O₃;
implementing a reduction operation, comprising: using a fixed bed reactor to introduce carbon dioxide gas into the first inorganic composite powder material under a reaction condition, so as to reduce the carbon dioxide gas into a carbon component that is absorbed at a nickel atom end of the first inorganic composite powder material;
implementing a sintering operation, comprising: under a sintering condition, performing lattice rearrangement for the first inorganic composite powder material absorbed with the carbon component at the nickel atom end, so as to obtain a second inorganic composite powder material that is composed of the following chemical components: C: Na—Ni/Al₂O₃; and
implementing a grafting operation, comprising: respectively reacting a first functional ionic liquid and a second functional ionic liquid with a siloxane coupling agent, and having the first functional ionic liquid and the second functional ionic liquid grafted on the second inorganic composite powder material through the siloxane coupling agent, so as to obtain the multifunctional catalyst.

11. The method according to claim 10, wherein the first functional ionic liquid is an imidazole ionic liquid, and is at least one material selected from a group consisting of [C4mim][PF6], [C6mim][PF6], [C6mim][BF4], and [C8mim][PF6].

12. The method according to claim 11, wherein the second functional ionic liquid is a salt composed of a cation and an anion, the cation is at least one material selected from a group consisting of imidazolium cation, pyridium cation, quaternary phosphonium cation, and quaternary ammonium cation, and the anion is at least one material selected from a group consisting of CI⁻, Br⁻, I⁻, AlCl₄⁻, AlBr₄⁻, AlI₄⁻, CF₃COO⁻, CH₃COO⁻, CF₃SO₃⁻, SCN⁻, (CF₃SO₂)₂N⁻, (CF₃SO₂⁻)₃C⁻, and C₆H₄(OH)(COO⁻).

13. A method for using a multifunctional catalyst, comprising:
providing a polyester fabric that is a dyed polyester fabric;
providing a multifunctional catalyst that comprises a carrier, and a first functional ionic liquid and a second functional ionic liquid that are grafted on the carrier, wherein the carrier is an inorganic composite powder material that is composed of the following chemical components: C: Na—Ni/Al₂O₃;
mixing the polyester fabric, the multifunctional catalyst, and a chemical depolymerization solution so that the first functional ionic liquid decolorizes the polyester fabric and the second functional ionic liquid depolymerizes the polyester fabric, so as to obtain a depolymerized product after decolorization and depolymerization, wherein the depolymerized product contains bis(2-hydroxyethyl) terephthalate (BHET); and
separating the multifunctional catalyst from the BHET.
